(19) United States
(12) Patent Application Publication
(10) Pub. No.: US 2026/0256663 A1
(43) Pub. Date: Sep. 3, 2026

(54) Smart liposome cosmetic composition with skin condition improvement effect

(71) Applicant: The Founders Inc., Seoul (KR)

(72) Inventors: Sung Hee Jang, Chungcheongnam-do (KR); Ho Kyung Lee, Chungcheongnam-do (KR)

(21) Appl. No.: 19/540,885

(22) Filed: Feb. 16, 2026

(30) Foreign Application Priority Data
Feb. 19, 2025 (KR) 10-2025-0021482

(51) Int. Cl. A61K 8/14; A61K 8/37; A61K 8/55; A61K 8/60; A61K 8/64; A61Q 19/02; A61Q 19/08

(52) U.S. Cl. CPC A61K 8/14; A61K 8/375; A61K 8/553; A61K 8/602; A61K 8/64; A61Q 19/02; A61Q 19/08; A61K 2800/591

(57) ABSTRACT

The present disclosure relates to a liposome composition including an oil phase portion containing phospholipids; and an aqueous phase portion containing a skin active substance, a melanocyte-targeting peptide, and a surfactant, a cosmetic composition including the liposome composition as an active ingredient, and the like. The composition according to an aspect is characterized in that the melanocyte-targeting peptide can increase the delivery efficiency of skin active substances by targeting target cells including melanocytes, and optimize the stability and activity of liposomes according to a combination ratio of surfactants.

### CROSS-REFERENCE TO RELATED APPLICATION

This present application claims the benefit of priority to Korean Patent Application No. 10-2025-0021482, entitled “Smart liposome cosmetic composition with skin condition improvement effect,” filed on Feb. 19, 2025, in the Korean Intellectual Property Office, the entire disclosure of which is incorporated herein by reference.

### FIELD

The present disclosure relates to a liposome composition including an oil phase portion containing phospholipids; and an aqueous phase portion containing a skin active substance, a melanocyte-targeting peptide, and a surfactant, a cosmetic composition including the liposome composition as an active ingredient, and the like.

### BACKGROUND

Skin health and beauty are important elements in modern life, and cosmetic products that aim to improve various aspects of the skin, such as whitening, anti-oxidation, anti-aging, elasticity, moisturizing, and wrinkle improvement, have received great attention. Accordingly, the demand for maintaining healthy skin is rapidly increasing, and various functional cosmetics have been researched and developed to meet this demand.

Liposomes are a representative biocompatible skin delivery carrier mainly used in a drug delivery system (DDS), and are characterized by being able to carry both hydrophilic and hydrophobic substances, in which water-soluble ingredients are solubilized in an aqueous phase center and fat-soluble ingredients are solubilized in a phospholipid membrane. The liposomes may efficiently deliver active ingredients to the skin, which are advantageous to increase absorption and deliver the active ingredients into a deep layer of the skin, and may be used to improve various skin conditions, such as anti-oxidation, anti-aging, skin elasticity improvement, moisturizing, and wrinkle improvement as well as skin whitening. In particular, smart liposomes may be designed to specifically deliver active ingredients to target cells or to target cosmetic ingredients and release active ingredients based on external stimuli (e.g., pH, temperature, ionic concentration, etc.).

However, although conventional cosmetic products contain various active ingredients, the cosmetic products often have short-term effects due to ineffective delivery of the active ingredients to the skin or structural instability. Under the background, the present inventors developed liposomes composed in an optimal content to enhance skin absorption of active substances having skin regeneration, anti-aging, anti-oxidation, wrinkle improvement, elasticity enhancement, and skin whitening activities.

### SUMMARY

One aspect provides a liposome composition including an oil phase portion containing phospholipids; and an aqueous phase portion containing a skin active substance, a melanocyte-targeting peptide, and a surfactant.

Another aspect provides a cosmetic composition including the liposome composition.

Yet another aspect provides a skin external preparation composition including the liposome composition.

One aspect provides a liposome composition including an oil phase portion containing phospholipids; and an aqueous phase portion containing a skin active substance, a melanocyte-targeting peptide, and a surfactant.

As used herein, the term ‘liposome’ means a spherical or oval-shaped structure surrounded by a fine lipid membrane, and has a vesicle shape of a lipid bilayer or multilayer.

The lipid may be an amphiphilic lipid, specifically a phospholipid, and the phospholipid may be any one selected from the group consisting of lecithin, phosphatidylcholine, phosphatidic acid, phosphatidylserine, phosphatidyl glycerol, phosphatidylinositol, phosphatidylethanolamine, diphosphatidylglycerol, cardiolipin, plasmalogen, dicetyl phosphate, distearoylphosphatidylcholine, dioleoylphosphatidylethanolamine, dipalmitoylphosphatidylcholine, dipalmitoylphosphatidylethanolamine, dipalmitoyl phosphatidylserine, eleostearoyl phosphatidylcholine, eleostearoyl phosphatidylethanolamine, eleostearoyl phosphatidylserine, and any combination thereof.

Specifically, the ‘lecithin’ may be naturally produced as a diglyceride mixture of stearic acid, palmitic acid, and oleic acid, and the composition of fatty acids may vary depending on a source thereof. In an embodiment of the present disclosure, hydrogenated lecithin produced through controlled hydrogenation (hydrogen addition) of lecithin was used.

The phospholipids may be included in the range of 0.01 to 10 parts by weight, 0.01 to 5 parts by weight, 0.01 to 3 parts by weight, 0.01 to 1 part by weight, 0.05 to 10 parts by weight, 0.05 to 5 parts by weight, 0.05 to 3 parts by weight, 0.05 to 1 part by weight, 0.05 to 10 parts by weight, 0.1 to 10 parts by weight, 0.1 to 5 parts by weight, 0.1 to 3 parts by weight, 0.1 to 1 part by weight, 0.5 to 10 parts by weight, 0.5 to 5 parts by weight, 0.5 to 3 parts by weight, 0.5 to 1 part by weight, 1 to 10 parts by weight, 1 to 5 parts by weight, and 1 to 3 parts by weight, based on 100 parts by weight of the liposome composition.

The surfactant may be at least one selected from the group consisting of polyglyceryl-6 laurate, lauryl glucoside, myristyl glucoside, polyglyceryl-10 caprylate, polyglyceryl-3 methyl glucose distearate, glucolipid, lysolecithin, sorbitan stearate, glyceryl oleate, glyceryl stearate, polyoxyalkylene alkyl ether, polyoxyalkylene fatty acid ester, alkyl polyglucoside, sodium lauryl ether sulfate, stearyl trimethyl ammonium chloride, alkyl polyglyceryl ether, stearyl trimethyl ammonium chloride, lauryl trimethyl ammonium chloride, distearyl dimethyl-ammonium chloride, poly(N, N′dimethyl-3,5-methylenepiperidinium) chloride, cetylpyridinium chloride, alkyl tetraammonium salt, alkyl dimethyl benzylammonium salt, alkylisoquinolinium salts, dialkyl-morpholinium salts, polyamine fatty acid derivatives, amyl alcohol fatty acid derivatives, benzalkonium chloride and benzethonium chloride, and potassium cetyl phosphate, and specifically, any one selected from the group consisting of polyglyceryl-6 laurate, lauryl glucoside, myristyl glucoside, polyglyceryl-10 caprylate, and combinations thereof.

The surfactant may be included in a range of 0.001 to 1 part by weight, 0.001 to 0.5 parts by weight, 0.001 to 0.3 parts by weight, 0.001 to 0.1 parts by weight, 0.005 to 1 part by weight, 0.005 to 0.5 parts by weight, 0.005 to 0.3 parts by weight, 0.005 to 0.1 parts by weight, 0.01 to 1 part by weight, 0.01 to 0.5 parts by weight, 0.01 to 0.3 parts by weight, 0.01 to 0.1 parts by weight, 0.05 to 1 part by weight, 0.05 to 0.5 parts by weight, 0.05 to 0.3 parts by weight, and 0.05 to 0.1 parts by weight, based on 100 parts by weight of the liposome composition.

As used herein, the term ‘skin active substance’ may mean all substances that may impart useful activities to subjects, such as skin regeneration, anti-aging, anti-oxidation, wrinkle improvement, elasticity enhancement, and skin whitening activities, etc. Specifically, the skin active substance may be (i) an antioxidant ingredient, (ii) an anti-inflammatory or skin soothing ingredient, (iii) a whitening ingredient, or (iv) a moisturizing or skin barrier strengthening ingredient. Preferably, the antioxidant ingredient may be vitamins or derivatives thereof, polyphenol compounds or flavonoid compounds, specifically vitamin C (ascorbic acid), ascorbyl glucoside, 3-O-ethylascorbic acid, magnesium ascorbyl phosphate, tocopherol, coenzyme Q10, glutathione, carotenoids, anthocyanin, catechin, apigenin, luteolin, genistein, quercetin, curcumin, kaempferol, resveratrol, isoflavones or flavonoids. In addition, the anti-inflammatory or skin soothing ingredient may be plant-derived extracts, bisabolol, licorice extract, broussonetia extract or glycyrrhizin. The whitening ingredient may be arbutin, phenethyl resorcinol, tranexamic acid, potassium methoxysalicylate, or vitamin C and its derivatives. The moisturizing or skin barrier strengthening ingredient may be collagen or hyaluronic acid. In addition, the skin active substance may be one or more of the above-mentioned compounds or derivatives thereof, but is not particularly limited thereto.

Depending on a type of skin active substance carried in the liposome of the present disclosure, the specific efficacy of a cosmetic composition or skin external preparation to which the skin active substance is applied may vary, and the type and content of the carried skin active substance may be appropriately selected by those skilled in the art.

The skin active substance may be included in a range of 0.01 to 10 parts by weight, 0.01 to 5 parts by weight, 0.01 to 3 parts by weight, 0.01 to 1 part by weight, 0.05 to 10 parts by weight, 0.05 to 5 parts by weight, 0.05 to 3 parts by weight, 0.05 to 1 part by weight, 0.05 to 10 parts by weight, 0.1 to 10 parts by weight, 0.1 to 5 parts by weight, 0.1 to 3 parts by weight, 0.1 to 1 part by weight, 0.5 to 10 parts by weight, 0.5 to 5 parts by weight, 0.5 to 3 parts by weight, 0.5 to 1 part by weight, 1 to 10 parts by weight, 1 to 5 parts by weight, and 1 to 3 parts by weight, based on 100 parts by weight of the liposome composition.

The melanocyte-targeting peptide may be any one selected from the group consisting of NAP represented by an amino acid sequence set forth in SEQ ID NO:1, LK-514 represented by an amino acid sequence set forth in SEQ ID NO:2, γ-MSH represented by an amino acid sequence set forth in SEQ ID NO:3, and combinations thereof.

The melanocyte-targeting peptide is included in the liposome composition to effectively deliver the skin active substance included in the liposome composition to melanocytes, thereby enhancing the effect of the skin active substance.

Specifically, the melanocyte-targeting peptide targets melanocytes and may be Ac-Nle-DHfRWGK (NAP) (SEQ ID NO:1); (4-phenylbutyryl)-H-f(tBu)-RK (LK-514) (SEQ ID NO:2); YVMGHFRWDRFK (γ-MSH) (SEQ ID NO:3); and combinations thereof.

The melanocyte-targeting peptide may be included in a range of 0.001 to 0.9 parts by weight, 0.001 to 0.5 parts by weight, 0.001 to 0.3 parts by weight, 0.001 to 0.1 parts by weight, 0.005 to 0.9 parts by weight, 0.005 to 0.5 parts by weight, 0.005 to 0.3 parts by weight, 0.005 to 0.1 parts by weight, 0.01 to 0.9 parts by weight, 0.01 to 0.5 parts by weight, 0.01 to 0.3 parts by weight, 0.01 to 0.1 parts by weight, 0.05 to 0.9 parts by weight, 0.05 to 0.5 parts by weight, 0.05 to 0.3 parts by weight, and 0.05 to 0.1 parts by weight, based on 100 parts by weight of the liposome composition.

The melanocyte-targeting peptides NAP, LK-514 and γ-MSH are preferably included in a weight ratio of 0.5 to 5:0.5 to 5:0.5 to 5, and specifically, may be included in a weight ratio of 0.5 to 4:0.5 to 4:0.5 to 4; 0.5 to 3:0.5 to 3:0.5 to 3; 0.5 to 2:0.5 to 2:0.5 to 2; or 0.5 to 1.5:0.5 to 1.5:0.5 to 1.5. In addition, the melanocyte-targeting peptides may be included in a weight ratio of 0.6 to 4:0.6 to 4:0.6 to 4; 0.6 to 3:0.6 to 3:0.6 to 3; 0.6 to 2:0.6 to 2:0.6 to 2; or 0.6 to 1.5:0.6 to 1.5:0.6 to 1.5. Furthermore, the melanocyte-targeting peptides may be included in a weight ratio of 0.7 to 4:0.7 to 4:0.7 to 4; 0.7 to 3:0.7 to 3:0.7 to 3; 0.7 to 2:0.7 to 2:0.7 to 2; or 0.7 to 1.5:0.7 to 1.5:0.7 to 1.5. Furthermore, the melanocyte-targeting peptides may be included in a weight ratio of 0.8 to 4:0.8 to 4:0.8 to 4; 0.8 to 3:0.8 to 3:0.8 to 3; 0.8 to 2:0.8 to 2:0.8 to 2; or 0.8 to 1.5:0.8 to 1.5:0.8 to 1.5. Furthermore, the melanocyte-targeting peptides may be included in a weight ratio of 0.9 to 4:0.9 to 4:0.9 to 4; 0.9 to 3:0.9 to 3:0.9 to 3; 0.9 to 2:0.9 to 2:0.9 to 2; or 0.9 to 1.5:0.9 to 1.5:0.9 to 1.5. Furthermore, the melanocyte-targeting peptides may be included in a weight ratio of 0.95 to 4:0.95 to 4:0.95 to 4; 0.95 to 3:0.95 to 3:0.95 to 3; 0.95 to 2:0.95 to 2:0.95 to 2; or 0.95 to 1.5:0.95 to 1.5:0.95 to 1.5, and most preferably in a weight ratio of 1:1:1.

As mentioned above, the liposome composition includes a melanocyte-targeting peptide, a surfactant, a skin active substance, and phospholipids. At this time, the liposome composition may include 150 to 300 parts by weight of the surfactant, 200 to 600 parts by weight of the skin active substance, and 100 to 300 parts by weight of the phospholipids, based on 1 part by weight of the melanocyte-targeting peptide.

Preferably, the surfactant may be included in an amount of 160 to 280 parts by weight, 170 to 260 parts by weight, 180 to 240 parts by weight, 190 to 220 parts by weight or 195 to 210 parts by weight, and most preferably 202 parts by weight. At this time, the surfactant may be polyglyceryl-6 laurate, lauryl glucoside, myristyl glucoside, polyglyceryl-10 caprylate or combinations thereof, and in the case of the combinations, the four ingredients may be combined in a weight ratio of 45 to 55:25 to 35:15 to 25:0.5 to 2, preferably 50:30:20:1.

Preferably, the skin active substance may be included in an amount of 250 to 550 parts by weight, 300 to 500 parts by weight, 350 to 450 parts by weight, 380 to 420 parts by weight, or 390 to 410 parts by weight, and most preferably 400 parts by weight. At this time, the skin active substance may be an antioxidant, such as ascorbic acid.

Preferably, the phospholipids may be included in an amount of 120 to 280 parts by weight, 140 to 260 parts by weight, 160 to 240 parts by weight, 180 to 220 parts by weight or 190 to 210 parts by weight, and most preferably 200 parts by weight. At this time, the phospholipids may be lecithin or hydrogenated lecithin.

Most preferably, the liposome composition may include 202 parts by weight of the surfactant, 400 parts by weight of the skin active substance, and 200 parts by weight of the phospholipids, based on 1 part by weight of the melanocyte-targeting peptide.

The liposome composition may be used for delivering skin active substances to skin cells.

Another aspect is to provide a cosmetic composition for improving a skin condition, including the liposome composition. The same part as described above is also entirely applied to the cosmetic composition.

Specifically, the ‘improvement of the skin condition’ may mean at least one selected from the group consisting of skin regeneration, anti-aging, anti-oxidation, wrinkle improvement, elasticity enhancement, and skin whitening. Therefore, the cosmetic composition may be a cosmetic composition for skin regeneration, anti-aging, anti-oxidation, wrinkle improvement, elasticity enhancement, or skin whitening.

As used herein, the term ‘skin regeneration’ refers to a series of reactions in which skin tissue is reconstructed in response to skin aging or damage caused to the skin, and specifically, the skin regeneration may be performed by differentiation, proliferation, and migration of keratinocytes.

As used herein, the term ‘anti-aging’ refers to all actions that prevent functional degeneration due to aging of skin cells and tissues, and ‘anti-oxidation’ refers to the inhibition of oxidative stress and cell oxidation caused by free radicals or reactive oxygen species induced by ultraviolet rays, etc., and may mean reducing damage to skin cells caused by removing free radicals or reactive oxygen species. As used herein, the term ‘skin whitening’ means making the skin white, and specifically, may mean alleviating or improving various pigmentations, including freckles and blemishes, caused by excessive synthesis of melanin or melanin deposition.

As used herein, the term ‘improvement of skin wrinkles’ means all actions that reduce wrinkles caused by a decrease in collagen fibers or a decrease in collagen production in the skin or decrease the rate of occurrence of the wrinkles, and the ‘elasticity enhancement’ may mean all actions that increase the elasticity and resilience of the skin by increasing the absorption or production of collagen within the skin.

The cosmetic composition may be prepared in various forms, and for example, the composition may be prepared in any one formulation selected from the group consisting of toner (skin lotion), skin softener, skin toner, astringent, lotion, milk lotion, moisture lotion, nourishing lotion, massage cream, nourishing cream, moisture cream, hand cream, foundation, essence, nourishing essence, pack, soap, cleansing foam, cleansing lotion, cleansing cream, body lotion, body cleanser, suspension, gel, powder, paste, mask pack or sheet, and aerosol, and the composition in these formulations may be prepared according to a conventional method in the art. The cosmetic composition may further include preservatives, stabilizers, surfactants, solubilizers, moisturizers, emollients, ultraviolet absorbers, antiseptics, bactericides, antioxidants, pH adjusters, organic and inorganic pigments, fragrances, cooling agents or antiperspirants, etc. The mixing amount of the additional ingredients such as the moisturizer may be easily selected by those skilled in the art without impairing the purpose and effect of the present disclosure, and the mixing amount thereof may be 0.001 to 5 wt %, specifically 0.01 to 3 wt %, based on the total weight of the composition.

Yet another aspect is to provide a skin external preparation composition for improving a skin condition, including the liposome composition. The same part as described above is also applied to the skin external preparation composition.

The skin external preparation may be cream, gel, ointment, skin emulsifier, skin suspension, transdermal patch, drug-containing bandage, lotion, or a combination thereof. The skin external preparation may be appropriately mixed with ingredients commonly used in skin external preparations, such as cosmetics or medicines, for example, aqueous ingredients, oily ingredients, powder ingredients, alcohols, moisturizers, thickeners, ultraviolet absorbers, whitening agents, preservatives, antioxidants, surfactants, fragrances, colorants, various skin nutrients, or combinations thereof, as needed.

In addition, the skin external preparation may be appropriately mixed by those skilled in the art with metal sequestrants such as disodium edetate, trisodium edetate, sodium citrate, sodium polyphosphate, sodium metaphosphate, and gluconic acid; caffeine, tannin, verapamil, licorice extract, glabridin, hot water extract of the fruit of Kalin, various herbal medicines, drugs such as tocopherol acetate, glycyrrhizic acid, tranexamic acid and derivatives or salts thereof, vitamin C, magnesium ascorbic acid phosphate, ascorbic acid glucoside, arbutin, kojic acid, and sugars such as glucose, fructose, and trehalose.

In another aspect, the present disclosure provides a method for improving a skin condition, including administering to a subject in need thereof a liposome composition including an oil phase portion containing phospholipids; and an aqueous phase portion containing a skin active substance, a melanocyte-targeting peptide, and a surfactant.

Specifically, the ‘improvement of the skin condition’ may mean at least any one selected from the group consisting of skin regeneration, anti-aging, anti-oxidation, wrinkle improvement, elasticity enhancement, and skin whitening.

The improvement of the skin condition may be at least any one selected from the group consisting of skin regeneration, anti-aging, anti-oxidation, wrinkle improvement, elasticity enhancement, and skin whitening.

The liposome composition according to an aspect or the cosmetic composition including the liposome composition is characterized in that the melanocyte-targeting peptide may target targeted cells including melanocytes to increase the delivery efficiency of skin active substances, and optimize the stability and activity of the liposomes according to a combination ratio of surfactants.

### BRIEF DESCRIPTION OF THE DRAWINGS

FIG. 1 shows results of confirming changes in melanin production according to treatment with liposome compositions of Example and Comparative Examples.

FIG. 2 shows results of confirming tyrosinase inhibitory activity according to treatment with liposome compositions of Example and Comparative Examples.

FIG. 3 shows results of measuring the expression level of a whitening-related gene (TYR) according to treatment with liposome compositions of Example and Comparative Examples.

FIG. 4 shows results of measuring the expression level of a whitening-related gene (TRP1) according to treatment with liposome compositions of Example and Comparative Examples.

### DETAILED DESCRIPTION

Hereinafter, the present disclosure will be described in more detail through the following Examples. However, these Examples are only illustrative of the present disclosure, and the scope of the present disclosure is not limited to these Examples.

### Experimental Example 1: Preparation of Liposomes

Liposomes were prepared using a thin film method. Specifically, hydrogenated lecithin, which was required for liposome preparation, was dissolved in ethanol. Ethanol in which lipids were dissolved was added in a round-bottom flask and ethanol as a solvent was completely evaporated and removed using a rotary evaporator to prepare a thin lipid membrane.

Next, the flask was added with purified water and three types of melanocyte-specific melanocyte-targeting peptides (NAP: Ac-Nle-DHfRWGK (Ac: Acetyl, f: D-phenylalanine) (SEQ ID NO: 1); LK-514: (4-phenylbutyryl)-H-f(tBu)-RK (SEQ ID NO: 2); and γ-MSH: YVMGHFRWDRFK) (SEQ ID NO: 3)), polyglyceryl-6 laurate, lauryl glucoside, myristyl glucoside, polyglyceryl-10 caprylate, 1,2-hexanediol, ethylhexylglycerin, and ascorbic acid, and hydrated at room temperature. The melanocyte-targeting peptides NAP, LK-514 and γ-MSH were mixed in a weight ratio of 1:1:1. The final prepared solution was used to form liposomes with a size of 100 to 150 nm using a high-pressure homogenizer (Microfludizer). At this time, the prepared compositions of Example and Comparative Examples 1 and 2 were shown in Table 1 below.

TABLE 1

| | | Comparative | Comparative | |
|---|---|---|---|---|
| | | Example 1 | Example 2 | Example |
| Phase | INCI Name | (%) | (%) | (%) |
| A | Purified water | To 100 | To 100 | To 100 |
| | Targeting peptide | 0 | 1.0 | 0.005 |
| | Polyglyceryl-6 laurate | 0.5 | 0.5 | 0.5 |
| | Lauryl glucoside | 0.3 | 0.3 | 0.3 |
| | Myristyl glucoside | 0.2 | 0.2 | 0.2 |
| | Polyglyceryl-10 caprylate | 0.01 | 0.01 | 0.01 |
| | Ascorbic acid | 2 | 2 | 2 |
| | 1,2-hexanediol | 2 | 2 | 2 |
| | Ethylhexylglycerin | 0.05 | 0.05 | 0.05 |
| B | Ethanol | 80 | 80 | 80 |
| | Hydrogenated lecithin | 1 | 1 | 1 |

### Experimental Example 2: Confirmation of Whitening Effect of Liposome Composition

### 2-1. Confirmation of Melanin Production

To confirm changes in melanin production according to treatment with a liposome composition, B16F0 cells were inoculated in a 6-well plate at 1×10⁵ cells/well using a DMEM (no phenol) medium supplemented with 10% FBS. After stabilizing the cells for 24 hours, the cells were treated with α-melanocyte stimulating hormone (α-MSH) and the compositions of Example and Comparative Examples 1 and 2 at different concentrations and incubated for 72 hours. After 72 hours of incubation, the medium was removed and washed with PBS to acquire the cells, and melanin was dissolved by treating 200 μl of a NaOH (1 N NaOH+10% DMSO) solution at 60° C. for 30 minutes, and then the absorbance was measured at 475 nm using a microplate reader. The compositions of Example and Comparative Examples 1 and 2 prepared in Experimental Example 1 were treated at a concentration of 1% or 3%, respectively, and as a positive control group, arbutin was treated at a concentration of 0.0025%.

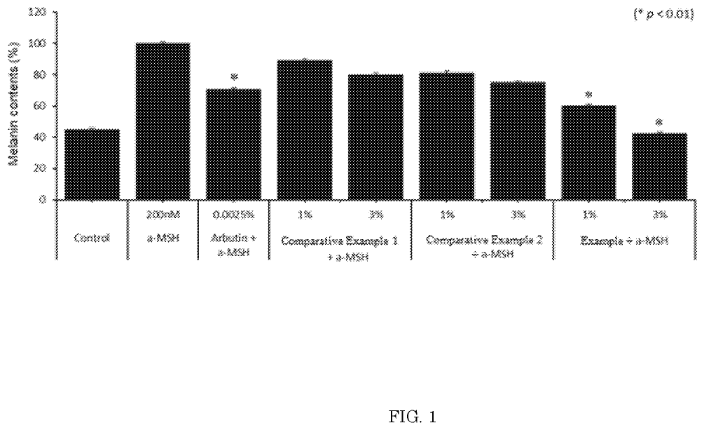

As a result, as shown in FIG. 1, it was shown that the liposome composition of Example significantly reduced the melanin production in the B16F0 cells compared to Comparative Examples 1 and 2, and the melanin production was decreased depending on a treatment concentration of the composition of Example. Through this, it was confirmed that the liposome composition of Example had excellent delivery efficiency of active ingredients to the target cells by including an appropriate amount of melanocyte-targeting peptide.

### 2-2. Confirmation of Tyrosinase Inhibitory Activity

To confirm the tyrosinase inhibitory activity according to treatment with a liposome composition, 10 μl of tyrosinase from mushrooms was reacted with a 0.1 M sodium phosphate buffer (pH 6.5). Additionally, L-tyrosine and the compositions of Example and Comparative Examples 1 and 2 diluted to each concentration were added, reacted for 5 minutes, and then the absorbance was measured using a microplate reader at a wavelength of 490 nm. As a positive control, 500 μg/ml of arbutin was treated.

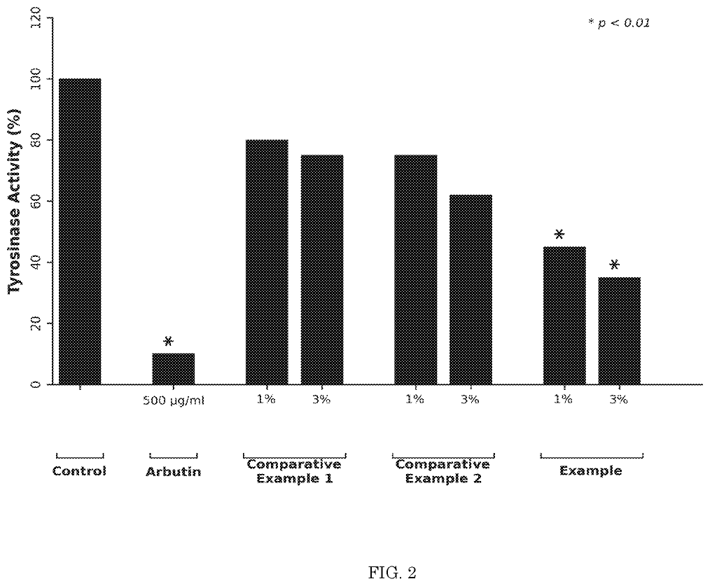

As a result, as shown in FIG. 2, it was shown that the liposome composition of Example significantly reduced the tyrosinase activity involved in melanin biosynthesis compared to Comparative Examples 1 and 2, and the tyrosinase inhibitory activity was increased depending on a treatment concentration of the composition of Example. From this, it was confirmed that the liposome composition of Example had excellent delivery efficiency of the active ingredients to the target cells by including an appropriate amount of melanocyte-targeting peptide.

### 2-3. Confirmation of Expression Levels of Whitening-Related Genes TYR and TRP1

To confirm the expression levels of whitening-related genes TYR and TRP1 according to treatment with a liposome composition, B16F0 cells were inoculated in a 6-well plate at 1×10⁵ cells/well using a DMEM (no phenol) medium supplemented with 10% FBS. After stabilizing the cells for 24 hours, the cells were treated with α-melanocyte stimulating hormone (α-MSH) and the compositions of Example and Comparative Examples 1 and 2 prepared in Experimental Example 1 at each concentration of 1% or 3% and incubated for 48 hours. Total RNA was extracted from the incubated cells using Trizol, and then the expression levels of TYR and TRP1 genes were confirmed through cDNA synthesis and real-time PCR. As a positive control, 200 μg/ml of arbutin was treated.

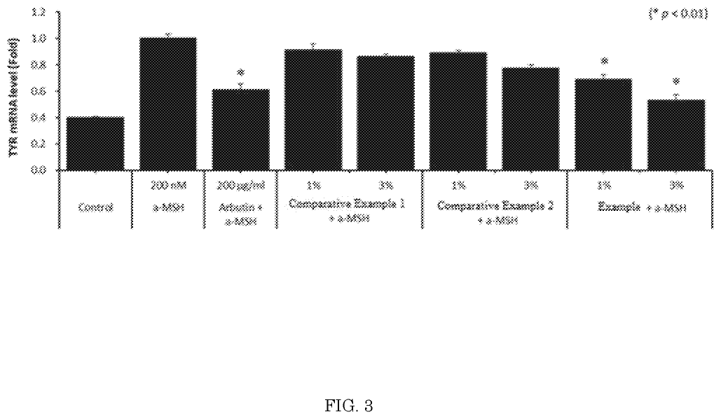

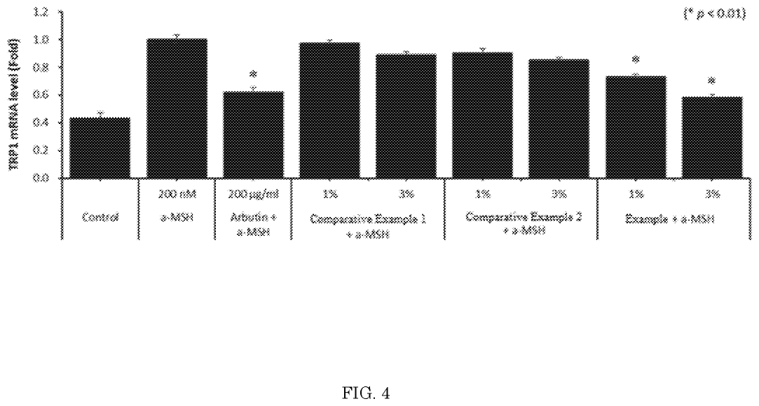

As a result, as shown in FIGS. 3 and 4, it was shown that the liposome composition of Example significantly reduced the expression levels of whitening-related genes TYR and TRP1 compared to Comparative Examples 1 and 2, and the expression levels of TYR and TRP1 were further decreased depending on a treatment concentration of the composition of Example. From this, it was confirmed that the liposome composition of Example had excellent delivery efficiency of the active ingredients to the target cells by including an appropriate amount of melanocyte-targeting peptide.

The aforementioned description of the present disclosure is to be exemplified, and it can be understood by those skilled in the art that the technical spirit or required features of the present disclosure can be easily modified in other detailed forms without changing. Therefore, it should be appreciated that the embodiments described above are illustrative in all aspects and are not restricted.

What is claimed is:

1. A liposome composition comprising an oil phase portion containing phospholipids; and an aqueous phase portion containing a skin active substance, a melanocyte-targeting peptide, and a surfactant.

2. The liposome composition of claim 1, wherein the phospholipid is any one selected from the group consisting of lecithin, phosphatidylcholine, phosphatidic acid, phosphatidylserine, phosphatidyl glycerol, phosphatidylinositol, phosphatidylethanolamine, diphosphatidylglycerol, cardiolipin, plasmalogen, dicetyl phosphate, distearoylphosphatidylcholine, dioleoylphosphatidylethanolamine, dipalmitoylphosphatidylcholine, dipalmitoylphosphatidylethanolamine, dipalmitoyl phosphatidylserine, eleostearoyl phosphatidylcholine, eleostearoyl phosphatidylethanolamine, eleostearoyl phosphatidylserine, and combinations thereof.

3. The liposome composition of claim 2, wherein the lecithin is hydrogenated lecithin.

4. The liposome composition of claim 1, wherein the melanocyte-targeting peptide is any one selected from the group consisting of NAP represented by an amino acid sequence set forth in SEQ ID NO: 1, LK-514 represented by an amino acid sequence set forth in SEQ ID NO: 2, γ-MSH represented by an amino acid sequence set forth in SEQ ID NO: 3, and combinations thereof.

5. The liposome composition of claim 4, wherein the melanocyte-targeting peptide is included in an amount of 0.001 to 0.9 parts by weight based on 100 parts by weight of the liposome composition.

6. The liposome composition of claim 4, wherein the NAP, LK-514 and γ-MSH are included in a weight ratio of 0.5 to 5:0.5 to 5:0.5 to 5.

7. The liposome composition of claim 1, wherein the surfactant is any one selected from the group consisting of polyglyceryl-6 laurate, lauryl glucoside, myristyl glucoside, polyglyceryl-10 caprylate, and combinations thereof.

8. The liposome composition of claim 1, wherein the skin active substance is at least one compound or derivatives thereof selected from the group consisting of vitamins or derivatives thereof, polyphenol compounds or flavonoid compounds, licorice extract, broussonetia extract, bisabolol, glycyrrhizin, arbutin, phenethyl resorcinol, tranexamic acid, potassium methoxysalicylate, collagen, hyaluronic acid, and combinations thereof.

9. The liposome composition of claim 1, wherein the liposome composition is used for delivering the skin active substances to skin cells.

10. A cosmetic composition for improving a skin condition, comprising the liposome composition of claim 1 as an active ingredient.

11. The cosmetic composition for improving the skin condition of claim 10, wherein the improvement of the skin condition is at least any one selected from the group consisting of skin regeneration, anti-aging, anti-oxidation, wrinkle improvement, elasticity enhancement, and skin whitening.

12. A skin external preparation composition for improving a skin condition, comprising the liposome composition of claim 1 as an active ingredient.

13. A method for improving a skin condition, comprising administering to a subject in need thereof a liposome composition comprising an oil phase portion containing phospholipids; and an aqueous phase portion containing a skin active substance, a melanocyte-targeting peptide, and a surfactant.

14. The method for improving the skin condition of claim 13, wherein the liposome composition is a cosmetic composition or skin external preparation composition.

15. The method for improving the skin condition of claim 13, wherein the improvement of the skin condition is at least any one selected from the group consisting of skin regeneration, anti-aging, anti-oxidation, wrinkle improvement, elasticity enhancement, and skin whitening.
